(19) United States
(12) Patent Application Publication
(10) Pub. No.: US 2020/0194109 A1
(43) Pub. Date: Jun. 18, 2020

(54) DIGITAL IMAGE RECOGNITION METHOD AND ELECTRICAL DEVICE

(71) Applicant: METAL INDUSTRIES RESEARCH & DEVELOPMENT CENTRE, KAOHSIUNG (TW)

(72) Inventors: Jian-Jia ZENG, KAOHSIUNG CITY (TW); Sheng-Hong YANG, KAOHSIUNG CITY (TW); Bo-Wei PAN, KAOHSIUNG CITY (TW)

(21) Appl. No.: 16/224,770

(22) Filed: Dec. 18, 2018

(51) Int. Cl. G16H 30/40; G06F 3/0484; G06K 9/00; G06K 9/62; H04L 29/08

(52) U.S. Cl. CPC G16H 30/40; G06F 3/04845; G06K 2209/05; G06K 9/6253; H04L 67/36; G06K 9/00973

(57) ABSTRACT

An image recognition method is provided and includes: decoding a digital image file to obtain a digital image; providing, by a front end application, a user interface so that a user interacts with the user interface through a browser, in which the user interface renders the digital image; receiving an image editing operation corresponding to the digital image through the user interface; obtaining characteristic information corresponding to a sample region of the digital image from the digital image file according to the image editing operation; transmitting, by the front end application, the characteristic information to a server to perform an image recognition procedure.

### BACKGROUND

### Field of Invention

The present invention relates to a digital image recognition method. More particularly, the present invention relates to a method providing an image recognition service through a browser.

### Description of Related Art

In recent years, the market for digital medical image recognition is growing rapidly. Recognizing medical images through artificial intelligence technology can solve problems of expensive human labor. Unlike general digital images, the medical images do not have a universal file format, and therefore different vendors may use different file formats. In addition, a file may contain many different magnification images and some metadata. Therefore, how to provide a convenient service platform to browse, edit or recognize medical images is an issue of concern to those skilled in the field.

### SUMMARY

Embodiments of the present invention provide a digital image recognition method for an electrical device. The digital image recognition method includes: decoding a digital image file to obtain a digital image; providing, by a front-end application, a user interface rendering the digital image for a user to interact with the user interface through a browser; receiving, through the user interface, at least one image edit operation corresponding to the digital image; obtaining characteristic information corresponding to at least one sample region of the digital image from the digital image file according to the at least one image edit operation; and transmitting, by the front-end application, the characteristic information corresponding to the at least one sample region to a server for performing an image recognition process.

In some embodiments, before the step of decoding the digital image file, the digital image recognition method further includes: pre-downloading, by the front-end application, the digital image file. The step of decoding the digital image file is performed by the front-end application.

In some embodiments, the step of decoding the digital image file is performed by a back-end application. The digital image recognition method further includes: downloading, by the front-end application, the digital image.

In some embodiments, the digital image recognition method further includes: recoding, by the front-end application, the at least one image edit operation in an off-line state, and transmitting the at least one image edit operation to a back-end server unit being in an on-line state.

In some embodiments, the at least one image edit operation includes extraction or annotation.

In some embodiments, the characteristic information includes an image size, a coordinate on a screen, a pointer, a bias, a number of layers or a layer location.

In some embodiments, the digital image recognition method further includes: decoding the digital image file according to the characteristic information to obtain a second digital image, wherein distortion of the second digital image is less than distortion of the digital image; and transmitting, by the front-end application, the second digital image to the server for performing the image recognition process.

From another aspect, embodiments of the invention provide an electrical device including a memory and a processor. The memory stores instruction executed by the processor to perform the digital image recognition method.

### BRIEF DESCRIPTION OF THE DRAWINGS

The invention can be more fully understood by reading the following detailed description of the embodiment, with reference made to the accompanying drawings as follows.

FIG. 1 is a schematic diagram illustrating a digital image recognition system in accordance with an embodiment.

FIG. 2 is a schematic diagram illustrating digital images with different magnification in accordance with an embodiment.

FIG. 3 is a schematic diagram of decoding the digital image file in accordance with an embodiment.

FIG. 4 is a schematic diagram of operations of the digital image recognition system in accordance with an embodiment.

FIG. 5 is a flow chart of a digital image recognition method in accordance with an embodiment.

### DETAILED DESCRIPTION

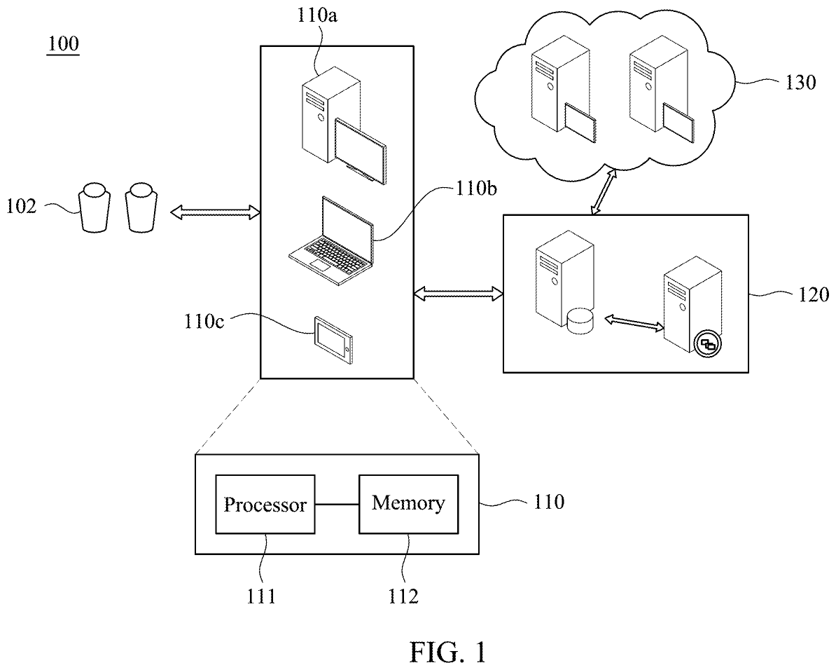

FIG. 1 is a schematic diagram illustrating a digital image recognition system in accordance with an embodiment. Referring to FIG. 1, a digital image recognition system 100 includes an electrical device 110, a back-end server 120, and a server 130. The electrical device 110 may be implemented as a personal computer 110a, a laptop 110b, a smart phone 110c or any other electrical device with computing capability. The electrical device 110 includes a processor 111 and a memory 112 storing instructions which are executed by the processor 111 to perform an image recognition method.

In detail, a user 102 can launch a browser on the electrical device 110 to access a particular website, and then a piece of program codes (also referred to a front-end application) is downloaded from the back-end server 120. The front-end application may be written by JavaScript, HyperText Markup Language (HTML), or other suitable language. The front-end application provides a user interface for the user 102 to interact with so that the user 102 can access a digital image file which will be described in detail below.

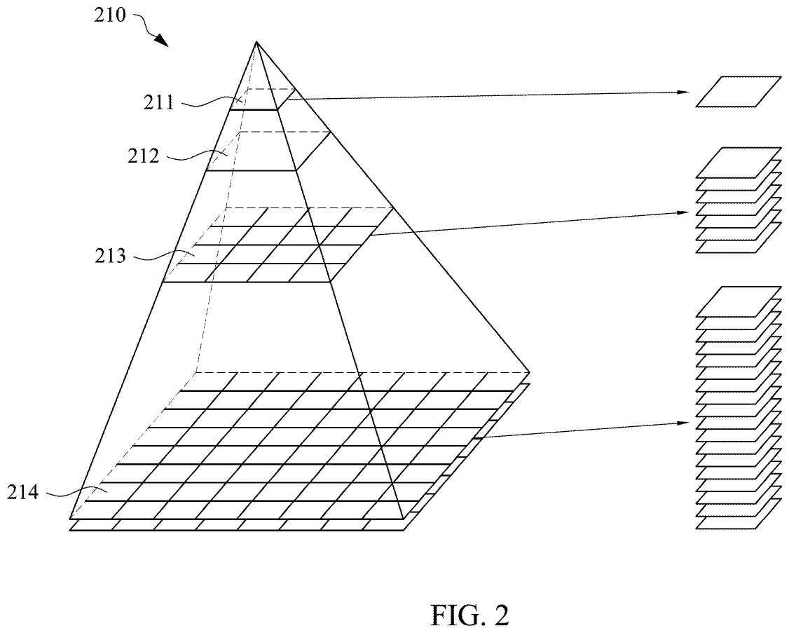

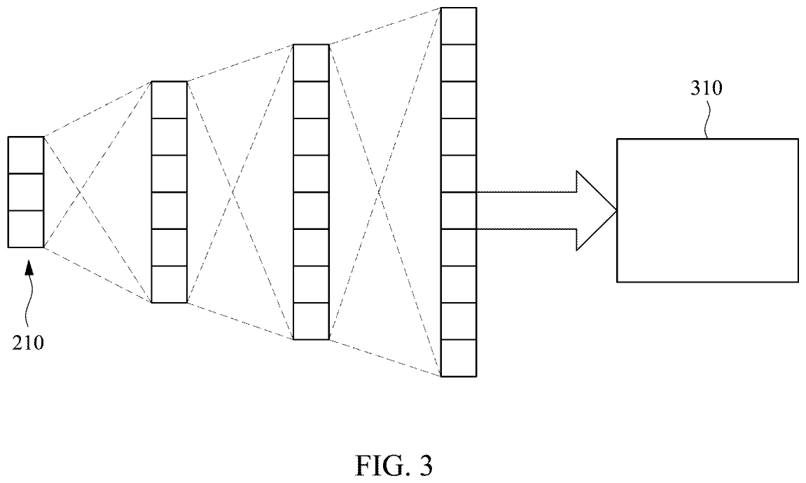

FIG. 2 is a schematic diagram illustrating digital images with different magnification in accordance with an embodiment. In some embodiments, the digital image file is related to medical images that generally have several different magnifications. As shown in FIG. 2, a digital image file 210 includes digital images 211-214 with different magnifications that are also referred to different layers in some embodiments. In addition, the digital image file 210 also includes some metadata such as image sizes, coordinates on a screen, pointers, bias in the file, the number of the layers, or layer locations etc. which is not limited in the invention. Therefore, the digital image file 210 has to be decoded first. FIG. 3 is a schematic diagram of decoding the digital image file in accordance with an embodiment. In some embodiments, the filename extension of the digital image file 210 may be “.mrxs”, “.svs”, “.scn”, or any other possible filename extension which is not limited in the invention. The digital image file 210 is typically a compressed file, and therefore one or more decoding process has to be executed to obtain the digital images. The user can review any one of the digital images. A digital image 310 is taken as an example for description.

Referring to FIG. 1 and FIG. 3, there are two ways to decode the digital image file 210. In the first way, the digital image file 210 is decoded by a back-end application on the back-end server 120, and then the front-end application downloads the decoded digital image 310. In the second way, the front-end application downloads the digital image file 210 and decodes the digital image file 210 by itself. In some embodiments, the front-end application may utilize graphics processing units (GPU) to perform parallel computation in order to speed up the decoding process. The invention is not limited to which of the above ways is adopted to decode the digital image file 210.

The user 102 can view the digital image 310 through the aforementioned browser. Note that the digital image 310 rendered by the browser may have distortion and problems of color bias due to the decoding process. For example, bit depth of each pixel of the original images of the digital image file 210 may be more than 8, but bit depth of each pixel of the digital image 310 may be only 8. Therefore, the digital image seen by the user is not identical to the original data of the digital image file 210. Next, the user 102 may perform at least one image edit operation, such as extraction or annotation, on the digital image 310 through the user interface of the browser to identify a portion to be recognized (also referred to a sample region). Then, the browser (or the front-end application) obtains characteristic information corresponding to the sample region of the digital image 310 from the digital image file 210. The characteristic information may include grey levels, image size, coordinates on a screen, pointers, bias, the number of the layers, or layer locations. For example, the image edit operation is used to clip a cell portion from the digital image 310. The browser obtains characteristic information of the cell portion from a respective layer and respective image data from the digital image file 210. Note that the front-end application does not extract the required portion from the rendered digital image 310 because it may produce wrong recognition result. Next, the front-end application transmits the characteristic information to the back-end server 120. The back-end server 120 may transmit the characteristic information to the server 130 for performing an image recognition process such as determining whether a biological tissue in the image is abnormal, or detecting a particular object in the image, or performing segmentation. The content of the image recognition process is not limited in the invention. In some embodiments, the image recognition process may be provided by the back-end server 120. In other words, the back-end server 120 and the server 130 are just for illustration, and they may be implemented as or integrated to more or less servers in some embodiments.

In some embodiments, after the image edit operation of the user is obtained, the front-end application decodes the digital image file 210 according to the characteristic information of the sample region to obtain another digital image (not shown) which has less distortion than that of the digital image 310. The distortion may be mean square error, absolute error, or any other index to measure image distortion, which is not limited in the invention. For example, after the user clips the cell portion on the browser, the front-end application will re-decode the data of the digital image file 210 corresponding to the cell portion to obtain another cell image with less distortion (e.g. with the same bit depth with that of the original image in the digital image file 210). After the digital image with less distortion is obtained, this digital image is transmitted by the front-end application to the server 130 for performing the image recognition process.

In some embodiments, when the user performs the image edit operation, the electrical device 110 is in an off-line state, and therefore the front-end application will record the image edit operation that occurs in the off-line state. The front-end application will transmit the image edit operation to the back-end server 120 batch by batch when the electrical device 110 is in an on-line state. For example, the user selects multiple image portions to be recognized in the off-line state. The sizes, locations, and layers of the selected image portions are recorded in a file such as Cookie of the browser. In the on-line state, the front-end application obtain corresponding characteristic information from the digital image file 210 according to the Cookie, and transmits the characteristic information to the back-end server 120 or the server 130 for the image recognition process.

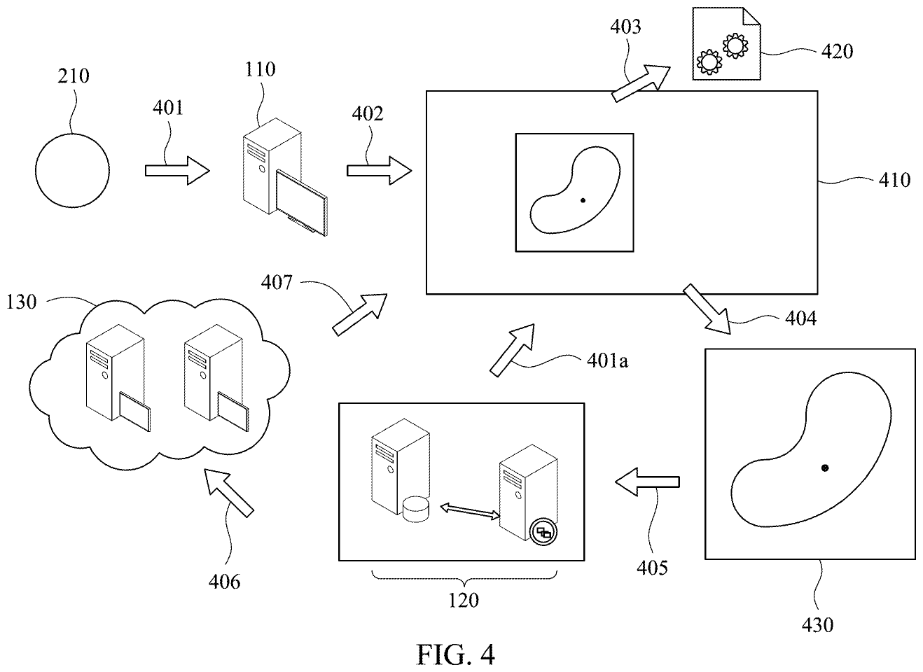

FIG. 4 is a schematic diagram of operations of the digital image recognition system in accordance with an embodiment. Two decoding ways are both illustrated in FIG. 4. In step 401, the front-end application of the electrical device 110 decodes the digital image file 210. In some embodiments, the digital image file 210 is pre-downloaded in the electrical device 110, and therefore the step 401 is performed in the off-line state. Next, in step 402, the front-end application renders a digital image 410 on the browser for the user to view and edit. In step 403, the image edit operation performed by the user is stored in a file 420 in the off-line state.

On the other hand, in the other decoding way, the back-end application in the back-end server 120 decodes the digital image file 210 in step 401a, and then the decoded digital image is transmitted to the electrical device 110 to be rendered in the browser as the digital image 410. Note that either step 401 or step 401a is performed.

In step 404, the front-end application obtains corresponding characteristic information 430 (shown as a cell portion herein) from the digital image file 210 according to the image edit operation of the user. In step 405, the front-end application transmits the characteristic information 430 to the back-end server 120. The back-end server 120 can perform the image recognition process by itself, or transmits the characteristic information 430 to the server 130 for the image recognition process (step 406). After the server 130 performs the image recognition process, in step 407, the recognition result is transmitted to the front-end application, or is transmitted to the back-end server 120 which transfers the recognition result to the front-end application in some embodiments. Finally, the user can check the recognition result.

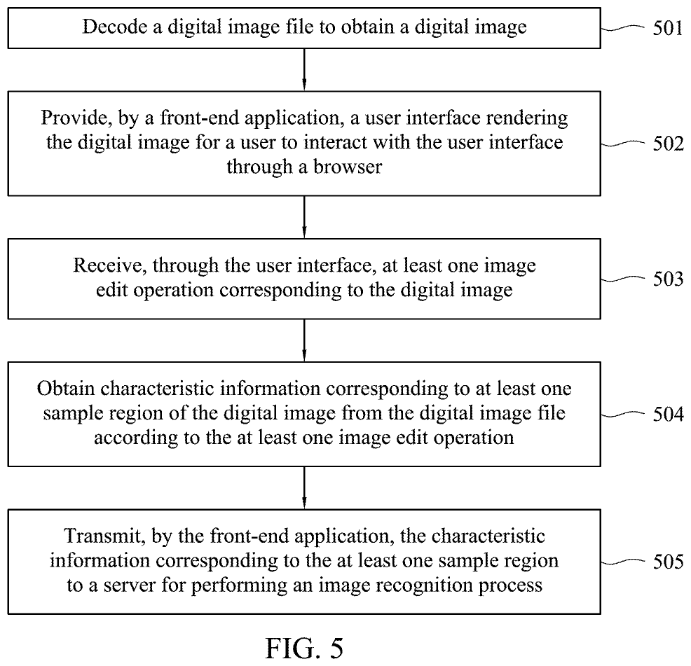

FIG. 5 is a flow chart of a digital image recognition method in accordance with an embodiment. Referring to FIG. 5, in step 501, a digital image file is decoded to obtain a digital image. In step 502, a user interface rendering the digital image is provided by a front-end application for a user to interact with the user interface through a browser. In step 503, receiving an image edit operation corresponding to the digital image through the user interface. In step 504, characteristic information corresponding to least one sample region of the digital image is obtained from the digital image file according to the image edit operation. In step 505, the characteristic information corresponding to the at least one sample region is transmitted by the front-end application to a server for performing an image recognition process. However, all the steps in FIG. 5 have been described in detail above, and therefore they will not be repeated. Note that the steps in FIG. 5 can be implemented as program codes or circuits, and the disclosure is not limited thereto. In addition, the method in FIG. 5 can be performed with the aforementioned embodiments, or can be performed independently. In other words, other steps may be inserted between the steps of the FIG. 5.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

1. A digital image recognition method for an electrical device, wherein the digital image recognition method comprises:
decoding a digital image file to obtain a digital image;
providing, by a front-end application, a user interface rendering the digital image for a user to interact with the user interface through a browser;
receiving, through the user interface, at least one image edit operation corresponding to the digital image;
obtaining characteristic information corresponding to at least one sample region of the digital image from the digital image file according to the at least one image edit operation; and
transmitting, by the front-end application, the characteristic information corresponding to the at least one sample region to a server for performing an image recognition process.

2. The digital image recognition method of claim 1, wherein before the step of decoding the digital image file, the digital image recognition method further comprises:
pre-downloading, by the front-end application, the digital image file, wherein the step of decoding the digital image file is performed by the front-end application.

3. The digital image recognition method of claim 1, wherein the step of decoding the digital image file is performed by a back-end application, and the digital image recognition method further comprises:
downloading, by the front-end application, the digital image.

4. The digital image recognition method of claim 1, further comprising:
recoding, by the front-end application, the at least one image edit operation in an off-line state, and transmitting the at least one image edit operation to a back-end server unit being in an on-line state.

5. The digital image recognition method of claim 1, wherein the at least one image edit operation comprises extraction or annotation.

6. The digital image recognition method of claim 1, wherein the characteristic information comprises an image size, a coordinate on a screen, a pointer, a bias, a number of layers or a layer location.

7. The digital image recognition method of claim 1, further comprising:
decoding the digital image file according to the characteristic information to obtain a second digital image, wherein distortion of the second digital image is less than distortion of the digital image; and
transmitting, by the front-end application, the second digital image to the server for performing the image recognition process.

8. (canceled)

9. An electrical device, comprising:
a memory storing a plurality of instructions; and
a processor executing the instructions to perform steps of:
decoding a digital image file to obtain a digital image;
providing, by a front-end application, a user interface rendering the digital image for a user to interact with the user interface through a browser;
receiving, through the user interface, at least one image edit operation corresponding to the digital image;
obtaining characteristic information corresponding to least one sample region of the digital image from the digital image file according to the at least one image edit operation; and
transmitting, by the front-end application, the characteristic information corresponding to the at least one sample region to a server for performing an image recognition process.
